(19) United States
(12) Patent Application Publication
(10) Pub. No.: US 2009/0326589 A1
(43) Pub. Date: Dec. 31, 2009

(54) HINGED PLATE FOR DYNAMIC STABILIZATION

(76) Inventors: Jeremy J. LEMOINE, Austin, TX (US); Brian J. BERGERON, Austin, TX (US)

Correspondence Address: Sprinkle IP Law Group, 1301 W. 25th Street, Suite 408, Austin, TX, 78705, US

(21) Appl. No.: 12/147,043

(22) Filed: Jun. 26, 2008

(51) Int. Cl. A61B 17/70

(52) U.S. Cl. 606/280; 606/246; 606/71; 606/297

(57) ABSTRACT

One embodiment provides a system which can include a pair of plates pivotably coupled to each other by a hinge. The plates can attach to posterior surfaces of vertebrae. The posterior surfaces can be on vertebral facets or can be exposed by removal of the facets. The hinge can be coupled to the plates in such a manner that the hinge is positioned adjacent to a center of rotation about which the vertebrae rotate relative to each other when the spine extends or flexes. The hinge can include a ball and socket, pin and pin hole, screw, etc and a sealing jacket. The system can include a piston for allowing the system to stretch and compress with the spine. Travel stops can be included in the hinge and piston. Multiple levels of the spine can be stabilized by systems with pairs of plates keyed to align with each other.

### TECHNICAL FIELD

Embodiments of the disclosure relate generally to spinal stabilization systems and methods and more particularly to dynamic spinal stabilization systems and methods.

### BACKGROUND

The human spine consists of segments known as vertebrae linked by intervertebral disks and held together by ligaments. There are 24 movable vertebrae—7 cervical, 12 thoracic, and 5 lumbar. Each vertebra has a somewhat cylindrical bony body (centrum), a number of winglike projections, and a bony arch. The bodies of the vertebrae form the supporting column of the skeleton. The arches are positioned so that the space they enclose forms the vertebral canal. It houses and protects the spinal cord, and within it the spinal fluid circulates. Ligaments and muscles are attached to various projections of the vertebrae.

The spine is subject to abnormal curvature, injury, infections, tumor formation, arthritic disorders, and puncture or slippage of the intervertebral disks. Injury or illness, such as spinal stenosis and prolapsed discs may result in intervertebral discs having a reduced disc height, which may lead to pain, loss of functionality, reduced range of motion, and the like. Scoliosis is one relatively common disease which affects the spinal column. It involves moderate to severe lateral curvature of the spine, and, if not treated, may lead to serious deformities later in life. One treatment involves surgically implanting devices to correct the curvature.

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures.

A spinal fixation system typically includes corrective spinal instrumentation that is attached to selected vertebra of the spine by screws, hooks, and clamps. The corrective spinal instrumentation includes spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Often, spinal fixation may include rigid (i.e., in a fusion procedure) support for the affected regions of the spine. Such systems limit movement in the affected regions in virtually all directions (e.g., in a fused region). More recently, so called “dynamic” systems have been introduced wherein the implants allow at least some movement (e.g., flexion, extension, lateral bending, or torsional rotation) of the affected regions in at least some directions.

### SUMMARY

One embodiment provides a posterior dynamic spinal stabilization system which can include a pair of plates and a hinge coupling the plates to each other. The plates can be shaped to conform to posterior surfaces of vertebrae for attachment to the vertebrae. The hinge can be positioned relative to the plates such that, when the plates are attached to the vertebrae, the hinge is generally adjacent a center of rotation about which the vertebrae rotate relative to each other. The hinge can include a ball and socket, a pin and pin hole, a spring, or other types of hinge mechanisms. A jacket can seal the hinge. The posterior vertebral surfaces, which the plates can attach to, can be on vertebral facets of the vertebrae, or can be surfaces exposed by removal of the vertebral facets. The plates can be keyed to each other so that multiple systems can be used in conjunction with each other to stabilize multiple levels of a spine. The keys on various plates can overlap and define apertures for attachment devices to attach pairs of plates to vertebra. Some systems can include pistons (with or without a travel stop) interposed between the hinge and one of the plates.

One embodiment provides a method of stabilizing a spine which can include selecting a pair of plates which are shaped to conform to posterior surfaces of vertebrae. The method can include causing the plates to be coupled by a hinge which allows them to pivot relative to each other. A position on the posterior surfaces can be selected at which the plates can be attached to the vertebrae in such a manner that the hinge will be generally adjacent to a center of rotation about which the vertebrae rotate when the spine flexes or extends. Vertebral facets can be removed from the vertebrae to expose the surfaces or the surfaces can be on the vertebral facets. The plates can have alignment keys to allow three or more plates to be used in conjunction with each other to stabilize the spine. The method can include selecting ball and socket, a pin, and a spring. A piston (with or without a travel limit) for coupling one of the plates to the hinge can also be selected.

One embodiment provides a dynamic spinal stabilization system which can include a pair of plates shaped to conform to vertebral facets of a pair of vertebrae and a hinge. The hinge can include a pin and pin hole and can be coupled to the plates in such a manner that when the plates are attached to the vertebrae, the hinge will be generally adjacent to a center of rotation about which the vertebrae rotate relative, to each other when the spine extends or flexes. A travel limit can also be included in the system to limit the relative travel between the plates.

Embodiments provide advantages over previously available dynamic spinal stabilization systems. Some embodiments provide spina) stabilization systems which move in a manner more closely corresponding to the anatomical movement of normal spines, in part, because the hinge can be generally adjacent to the center of rotation of affected vertebrae. Embodiments provide spinal stabilization systems with lower profiles and which can stabilize spines without protruding beyond the base area of the spinous processes.

Embodiments allow motion of stabilized spines to be tailored (with improved predictability of post-operative results) according to indications of the condition to be treated. For instance, in some embodiments relative rotation between affected vertebrae can be limited. Embodiments allow motion between affected vertebrae with single or multiple degrees of freedom as indicated by the conditions to be treated. Embodiments, provide dynamic spinal stabilization systems which do not require overcoming tensile forces to cause relative movement between affected vertebrae.

In methods of some embodiments, spinal stabilization systems can be attached to spines without bending, altering, modifying, etc. components (for instance, stabilization rods) of the systems thereby, among other benefits, eliminating cold-working of such components with attendant changes to their mechanical properties. By avoiding modifications to spinal stabilization system components during attachment, some embodiments avoid manually introducing inaccuracies into the configuration of previously available spinal stabilization systems.

Other features, advantages, and objects of the disclosure will be better appreciated and understood when considered in conjunction with the following description and the accompanying drawings.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a human axial skeleton,

FIG. 2 depicts one embodiment of a spinal stabilization system.

FIG. 3A depicts one embodiment of a spinal stabilization system attached to a spine.

FIG. 3A depicts one embodiment of a spinal stabilization system attached to a spine.

FIG. 4 depicts one embodiment of a spinal stabilization system.

FIG. 5 depicts one embodiment of a spinal stabilization system attached to a spine.

FIG. 6 depicts one embodiment of a spinal stabilization system.

FIG. 7 depicts one embodiment of a spinal stabilization system attached to a spine.

FIG. 8 depicts various embodiments of hinges for spinal stabilization systems.

FIG. 9A depicts one embodiment of a spinal stabilization system.

FIG. 9B depicts one embodiment of a spinal stabilization system.

FIG. 10 depicts a flowchart of one embodiment of a method for stabilizing a spine.

### DETAILED DESCRIPTION

The disclosure and the various features and advantageous details thereof are explained more fully with reference to the non-limiting embodiments detailed in the following description. Descriptions of well known starting materials, manufacturing techniques, components and equipment are omitted so as riot to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” “includes,” “including,” “has,” “having” or any other variation thereof, are intended to cover a non-exclusive inclusion. For example, a process, process, article, or apparatus that comprises a list of elements, is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of Ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in One embodiment”.

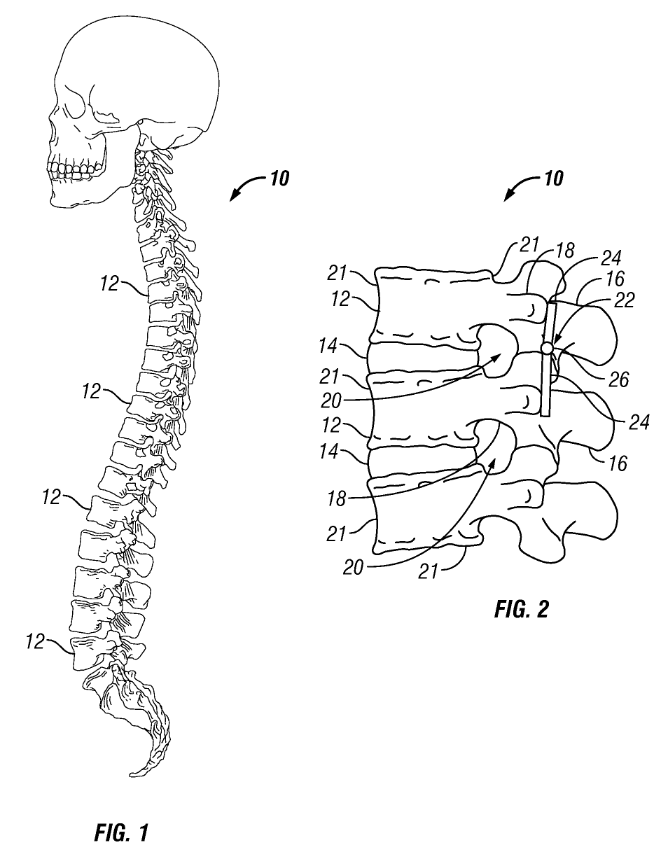

FIG. 1 depicts a human axial skeleton including a skull (composed of

numerous cranial bones (such as parietal bones, temporal bones, zygomatic bones, mastoid bones, maxilla bones, mandible bones, etc.) and spine 10 including numerous vertebrae 12, intervertebral discs, etc. As discussed previously, spine 10 carries loads imposed on the patient's body and generated by the patient. Vertebrae 12 cooperate to allow spine 10 to extend, flex, rotate, etc. under the influence of various muscles, tendons, ligaments, etc. attached to spine 10. Spine 10 can also cooperate with various muscles, tendons, ligaments, etc. to cause other anatomical features of the patient's body to move. However, certain conditions can cause damage to spine 10, vertebrae 12, intervertebral discs, etc. and can impede the ability of spine 10 to move in various manners. These conditions include, but are not limited to abnormal curvature, injury, infections, tumor formation, arthritic disorders, puncture, or slippage of the intervertebral disks, and injuries or illness such as spinal stenosis and prolapsed discs. As some of these conditions progress, or come into existence, various symptoms can indicate the desirability of stabilizing spine 10 or portions thereof. As a result of various conditions, the ability of the patient to move, with or without pain or discomfort, can be impeded. Based on such indications, medical personnel can recommend attaching one or more spinal stabilization systems to vertebrae 12 among other remedial actions such as physical therapy.

FIG. 2 depicts a side elevation view of a portion of spine 1 including, various vertebrae 12, inter-vertebral discs 14, spinous processes 16, transverse processes 17, and vertebral facets 18 of vertebrae 12, intravertebral area 20. FIG. 2 also depicts spinal stabilization system 22 including a pair of plates 24 and hinge 26 which can couple plates 24 together. Spinal stabilization system 22 can be attached to spine 10 with various attachment devices to correct conditions such as those discussed previously. As will be discussed with more particularity herein, spinal stabilization system 22 can be attached to various posterior surfaces of spine 10 while maintaining a profile which can remain anterior to the posterior ends of spinous processes 16.

It may be helpful at this juncture to briefly describe portions of vertebrae 18. For instance, potential attachment points for spinal stabilization system 22 can include transverse processes 17 (not shown), vertebral facets 18, various surfaces exposed by surgical personnel, etc. Spinous processes 16 and vertebral facets 17 (and other features of vertebrae 12) are boney structures. Spinous processes 16 and transverse processes 17 allow tendons, muscles, etc. to attach to spine 10 for movement of spine 10 and various anatomical structures which are attached to spine 10 or affected thereby in various mariners. These anatomical structures can include the patient's ribs, hips, shoulders, head, legs, etc. Spinous processes 16 extend generally in a posterior and slightly inferior direction from vertebrae 12. Transverse processes 17 are also boney structures and extend generally laterally from vertebrae 12 and allow muscles and tendons to attach to vertebra 18. Vertebral facets 18 join adjacent vertebrae 12 to each other while allowing motion there between by being in sliding contact with corresponding vertebral facets 18 of these adjacent vertebrae 12. During certain types of motion of spine 10 (such as flexing and extending) caused (or resisted) by various muscles, vertebrae 12 tend to rotate relative to each other about axes of rotation generally in intravertebral areas 20. Intravertebral areas 20 can be adjacent to and posterior to intervertebral discs 14 and substantially anterior to spinous processes 16 and vertebral facets 18. Since vertebral facets 18 allow vertebrae 18 to articulate about these axes of rotation, no, or little, reactionary forces or moments are generated by healthy spines 10 themselves during ordinary movements.

Previously available approaches to dynamically stabilizing spine 10 include attaching stabilization rods to spine 10 in manners causing the rods to lie posterior to spinous processes 16 and therefore anatomically distant from intravertebral areas 20 in which the vertebral axes of rotation lie. Since such previously available stabilization rods are distant from the vertebral axes of rotation they tend to generate reaction forces which resist movement of spine 10. Thus, as spine 10 extends or flexes, these previously available stabilization rods (being distant from vertebral axes of rotation in intravertebral areas 20) impede movement of spine 10. More particularly, the distances between vertebral axes of rotation and previously available stabilization rods can act as moment arms thereby generating moments and forces on spine 10. Therefore, spine 10 can cause reaction forces on the previously available spinal stabilization systems that can degrade the mechanical integrity and functioning of such spinal stabilization systems. Moreover, because such moments and forces (or their reactions) act on spine 10, spine 10 (and patient comfort and health) can be adversely affected). As a result, the range of motion and patient comfort could be adversely affected with previously available spinal stabilization approaches. In addition, the moments and forces generated due to the anatomically significant distances between vertebral axes of rotation and previously available spinal stabilization systems can degrade the mechanical integrity of and functioning of such spinal stabilization systems.

As FIG. 2 illustrates, one embodiment of spinal stabilization system 22 can be shaped and dimensioned to lay just posterior to arid adjacent to intravertebral area 20 (in which axis of rotation between various vertebrae 12 exist) when attached to spine 10. More particularly, plates 24 can be shaped to conform to the posterior surfaces of vertebral facets 18. Hinge 26 Can couple plates 24 to each other in such a manner that hinge 126 is positioned (when plates 24 are attached to vertebral facets 18) adjacent to (or within) intravertebral area 20. For instance, an offset, not shown, can be defined by plates 24 to position hinge 26 within area 20 Without departing from the scope of the disclosure. As spine 10 extends arid flexes, plates 24 can follow vertebral facets 18 with hinge 26 accommodating the anatomical movements of vertebrae 12. Thus, as spine 10 extends, hinge 26 allows plates 24 to pivot relative to each other in a manner generally conforming to anatomical movements of vertebrae 12. As spine 10 flexes, hinge 26 allows plates 24 to pivot relative to each other in the opposite direction (compared to when spine 10 extends) and in a manner generally conforming to anatomical movements of vertebrae 12. Because plates 24 and hinge 26 can follow vertebrae 18, moments and forces generated during such movements of spine 10 can be minimized. As a result, spine 10 and spinal stabilization system experience no, or little, additional forces and moments other than those that might be carried by spine 10 or generated by various muscles.

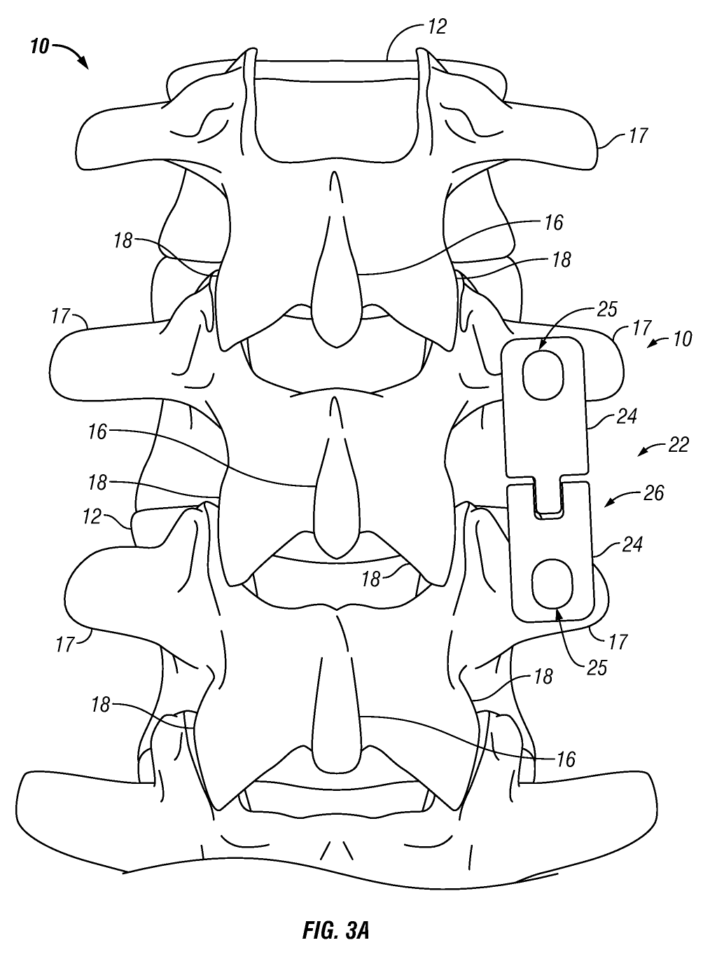

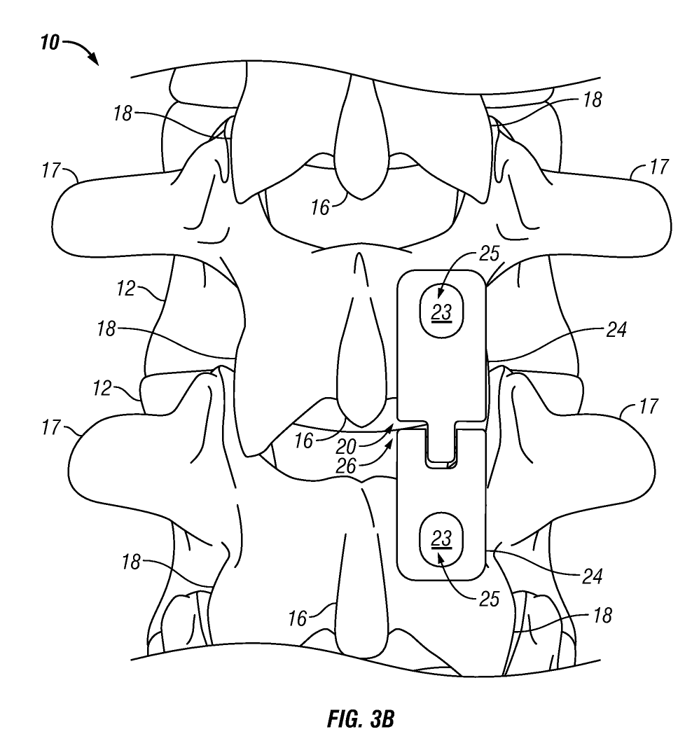

FIG. 3A illustrates one embodiment of spinal stabilization system 22 attached to posterior surfaces of vertebrae 18 and, more particularly, attached to vertebral facets 17 of vertebrae 12. Plates 24 are shown as attaching to adjacent vertebrae 12 with hinge 26 pitovably coupling plates 24 to each other. Thus, as vertebrae 12 rotate relative to one another about axes of rotation in intravertebral area 20 (not shown due to its location anterior to spinous process 16) spinal stabilization system 22 generally follows the anatomical movement of spine 10. FIG. 3A also illustrates attachment apertures 25 through which bone screws or other attachment devices can be driven to attach plates 24 to vertebrae 12. Attachment apertures can be generally circular in nature although they can be elongated to allow surgical personnel to adjust the position of plates 24 on vertebrae 18. In some embodiments, bone anchors and other attachment devices can be used to attach plates 24 to vertebrae 18 without departing from the scope of the disclosure. As illustrated by FIG. 3A, plates 24 can be generally oblong in shape when viewed from a direction posterior to spine 10. Plates 24 can be shaped and dimensioned to remain within the volume defined by the lateral extension of transverse processes 17 from vertebrae 12.

With reference to FIG. 3B, in one embodiment, vertebral facets 18 can be partially (or substantially completely) removed from vertebrae 12 to accommodate plates 24. FIG. 3B illustrates vertebral facets 18 having been partially removed from vertebrae 12 leaving exposed surfaces 23 for attachment of plates 24 thereto. For instance, FIG. 3B shows three vertebral facets 18 on the left side of vertebrae 12 but only two vertebral facets 18 on the right side of vertebrae 12. In FIG. 3B, vertebral facet 18 of middle vertebra 12 is shown as being removed for attachment of a particular plate 24 to vertebra 12. FIG. 3B also shows plates 24 attached to posterior surfaces 23 of vertebral facets 18 which were exposed when vertebral facet 18 was removed. Attaching plates 24 to such exposed posterior surfaces of vertebral facets 18 can allow placing plates 24 and hinge 26 closer (in a posterior-anterior direction) to intravertebral areas 20 (which can be just anterior to hinge 226 or coincident therewith) in which axes of rotation between vertebrae lies. Therefore, spinal stabilization system 22 can move in better conformity with anatomical movements of spine 10. In FIG. 3B, plates 24 are shown as being attached to relatively flat exposed surfaces 23 as opposed to on angled surfaces which various anatomical features of previously removed vertebral facets 18 possessed. Attaching plates 24 to such exposed surfaces 23 of vertebral facets 18 can allow for relatively improved predictability of post-operative results since plates 24 can be attached to vertebrae 12 at angles created by surgical personnel.

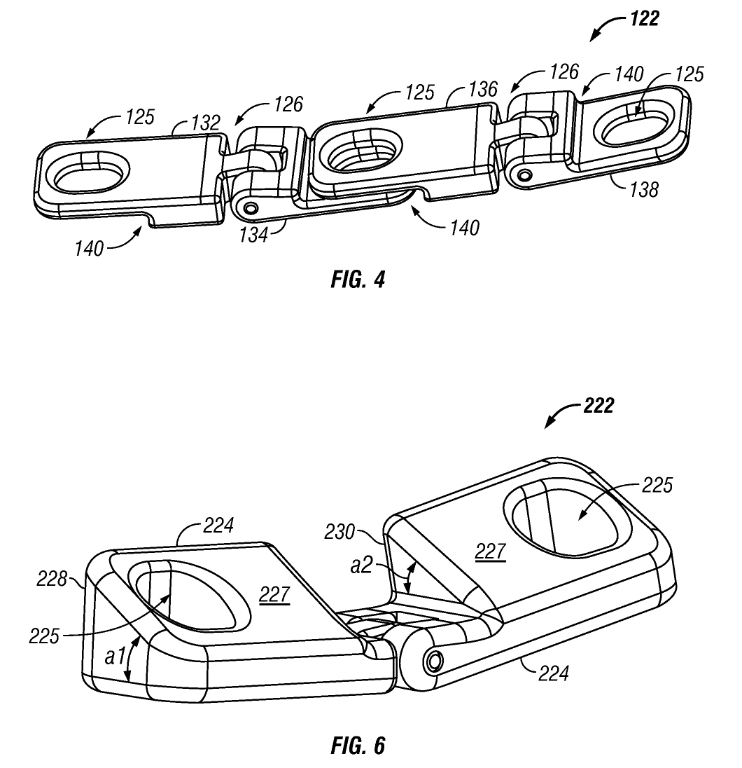

With reference now to FIG. 4, FIG. 4 illustrates one embodiment of a spinal stabilization system for stabilizing multiple levels of spine 10. Spinal stabilization system 122 includes two pairs of plates 132 and 134 and 136 and 138 and two hinges 126 pivotably coupling plates 132 and 134 and 136 and 138 together. Plates 132 and 138 on the superior and inferior ends of spinal stabilization system 122 can correspond to plates 24 of spinal stabilization system 22.

Plates 134 and 136 (in between plates 132 and 138) can include mating keys 140 such that plates 134 and 136 can be aligned with each other. Mating keys 140 can be configured so that plates 134 and 136 overlap sufficiently that attachment apertures 125 on plates 134 and 136 also align with each other thereby allowing one bone screw or other attachment device to attach plates 134 and 136 to a particular vertebra 12 of spine 10. Plates 132 and 138 on superior and inferior ends of spinal stabilization system 122 can include attachment apertures 125 corresponding to attachment apertures 25 (of FIG. 3A and 3B). Thus, surgical personnel may attach plates 134 arid 136 to a particular vertebra 12 and can attach plates 132 and 138 to appropriate vertebrae 12 to stabilize multiple levels of spine 10.

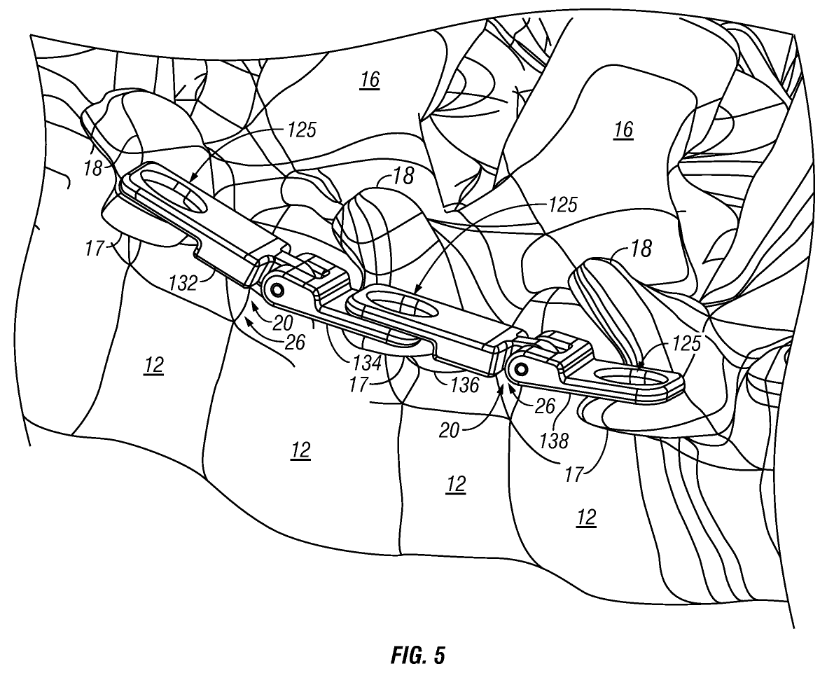

With reference now to FIG. 5, FIG. 5 illustrates spinal stabilization system 122 attached to spine 10 by various transverse processes 17. Plates 132, 134, 136, and 138 are shown lying along spine 10 in a superior to inferior direction. Bone screws (riot shown) Can attach plates 132, 134, 136, and 138 to transverse processes 17 via attachment apertures 125. FIG. 5 illustrates that spinal stabilization system 122 lies generally adjacent to base portions of vertebral facets 18 and generally adjacent to base portions of spinous processes 16 (when viewed looking medially toward spine 10). Hinges 126 can couple plates 132 and 134 and plates 136 and 138 to each other.

FIG. 5 also illustrates axes of rotation 121 about which vertebra 12 rotate relative to each other when spine 10 flexes or extends. As distances d1 and d2 illustrate, hinges 126 can lie adjacent to axes of rotation 121 with minimal distances d1 and d2 there between. In some embodiments, hinge 126 can be positioned with vertebral axis of rotation passing there through. When spine 10 extends or flexes, hinges 126 allow pairs of vertebrae 12 to rotate relative to each other about axes of rotation 121. Because distances d1 and d2 between hinges 126 and axes of rotation 121 can be minimized by embodiments, spinal stabilization system 122 can follow anatomical movements of spine 10 as spine 10 flexes and extends. Moreover, because of minimal distances d1 and d2, spinal stabilization system 122 imparts no, or little, reaction forces or moments on spine 10 and various portions thereof (such as vertebrae 12, transverse processes 17, vertebral facets 18, etc). Spinal stabilization system 122 can accommodate such forces and moments exerted on it by spine 10, in part, because of minimal distances d1 and d2 between hinges 126 and axes of rotation 121. Patient health and comfort can therefore be accommodated by spinal stabilization system 122. In addition, the mechanical integrity and functioning of spinal stabilization system 122 can be maintained.

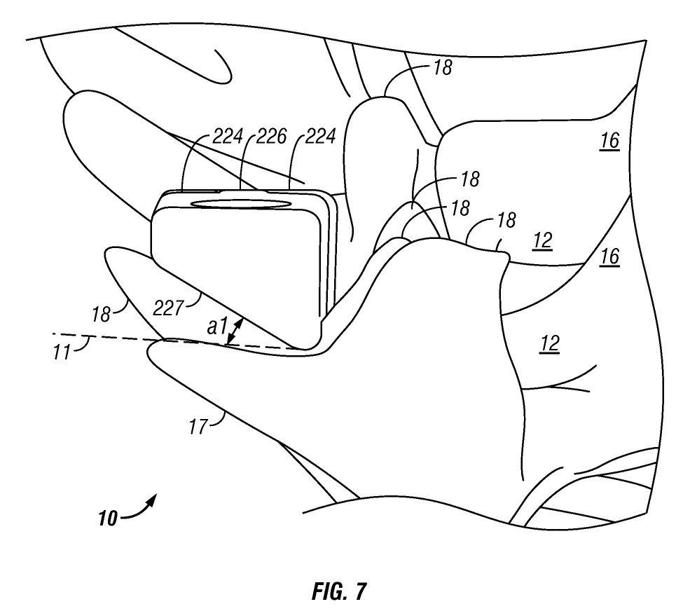

With reference now to FIG. 6, FIG. 6 illustrates a side elevation view of one embodiment of spinal stabilization system 22. FIG. 6 illustrates plates 224, hinge 226 pivotably coupling plates 224 together, and adapters 228 and 230 (which can be coupled to or formed integrally with plates 224). In FIG. 7, one particular plate 224 is shown as lying substantially in front of the hinge 226 and the other plate 224. Adapters 228 and 230 can be generally wedge shaped with anterior surfaces 227 angled at angles a1 and a2 relative to posterior surface 229. Posterior surface 229 can be shaped and dimensioned to generally follow the direction of spine 10 or the particular portion of spine 10 to which it can be attached. In some embodiments, posterior surface 229 of plate 224 can be flat and oriented (when plates 224 are attached to vertebrae 12) to be parallel to the direction of a particular portion of spine 10.

Angles a1 and a2 can, in part, define anterior surfaces 227 of plates 224. Angles a1 and a2 can correspond to angles a1 and a2 associated with selected transverse processes 17 of vertebrae 12 (see FIG. 7). While FIG. 6 illustrates adapters 228 and 230 having generally planar anterior surfaces 227, which when system 224 is attached to vertebrae 12 abut transverse processes 17, adapters 228 and 230 can be shaped to correspond to anatomical features of transverse processes 17 (see FIG. 7). Adapters 228 and 230 can adapt plates 224 for use at various levels along spine 10 as various patient symptoms may indicate. Adapters 228 and 230 can enhance system's 222 mechanical integrity and functioning without making modifications to transverse processes 18 desirable (except, perhaps, for the use of attachment devices to attach system 222 to transverse processes 17).

With continuing reference to FIG. 7, FIG. 7 illustrates one embodiment of spinal stabilization system 222 attached to spine 10 by transverse processes 17. FIG. 7 further illustrates that angles a1 associated with transverse process 17 vary with location along spine 10 (and between patients). Transverse processes 17 can extend from vertebrae 12 with their posterior surfaces being generally angled at angles such as a1. Angles a1 fend to increase with increasingly inferior positions of vertebrae 18. Particular plates 224 can include anterior surfaces 227 shaped to accommodate particular transverse processes 17 angles a1. Plates 224 can also be shaped to conform to other features of transverse processes 17 as surgical personnel may recommend based on features of transverse processes 17. Attaching plates 224 to transverse processes 17 as illustrated by FIG. 7 allows for attaching plates 224 to transverse processes 17 in a relatively simple fashion while minimizing disturbance of anatomical features of the patient and, more particularly, anatomical features of transverse processes 17.

FIG. 7 also illustrates vertebral axis of rotation 121 and hinge 226 axis of rotation 231. As distance d3 illustrates, hinge 226 axis of rotation 231 can lie adjacent to axes of rotation 121 with minimal distance d3 there between. When spine 10 extends or flexes, hinge 226 allows vertebrae 12 to rotate relative to each other about axis of rotation 121. Because distance d3 between hinge 126 and axis of rotation 121 can generally be minimized by embodiments, spinal stabilization system 222 can follow anatomical movements of spine 10 as spine 10 flexes and extends. Moreover, because of minimal distance d3, spinal stabilization system 222 imparts no, or little, reaction forces and moments on spine 10. Patient health and comfort can therefore be accommodated by spinal stabilization system 222. Spinal stabilization system 222 can also accommodate forces and moments exerted on it by spine 10, in part, because of minimal distance d3 between hinge 226 axis of rotation 221 and axis of rotation 121. As a result, the mechanical integrity and functioning of spinal stabilization system 222 can be maintained.

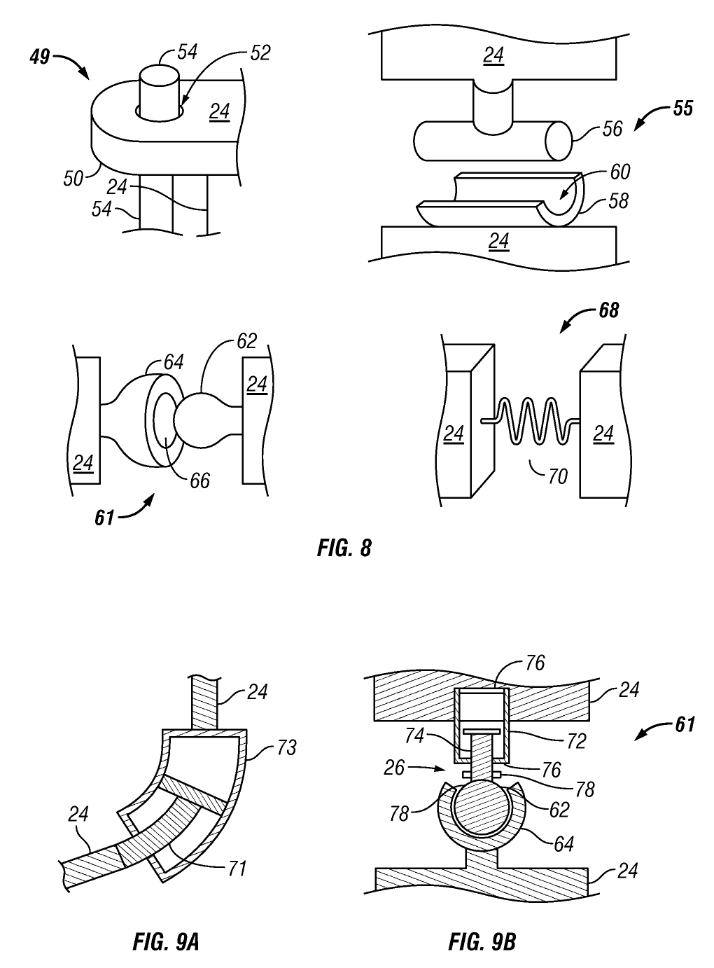

FIG. 8 illustrates several embodiments of hinges 49, 55, 61, and 68 for pivotably coupling plates 24 to each other. In one embodiment, hinge 49 includes a number of gussets 50 defining holes 52 in which pin 54 can be retained. Gussets 50 can be coupled to, or be formed integrally with plates 24 so that as spine 10 extends and flexes, plates 24 pivot about pin 54. Because plates 24 can attach to posterior surfaces 23 of vertebra 12, hinge 49 can allow spinal stabilization system 22 to follow anatomical movements of spine 10 as spine 10 flexes and extends.

FIG. 8 also illustrates hinge 55 of one embodiment. Hinge 55 can include pin 56 and gusset 58 which defines socket 60. Pin 56 can be coupled to, or formed integrally with, a particular plate 24 while gusset 58 can be coupled to, or formed integrally with, the other plate 24. Pin 56 can be fixed with regard to the particular plate 24 of spinal stabilization system 22 to which it is coupled. Socket 60 can correspond in shape to pin 56 and can capture pin 56 to pivotably couple plates 24 together. Hinge 55 can therefore allow plates 24 to follow anatomical movements of spine 10 as spine 10 flexes and extends.

FIG. 8 also illustrates hinge 61. Hinge 61 can include ball 62 and gusset 64. Gusset 64 can define socket 66 for receiving and perhaps capturing ball 62. Ball 62 can be formed integrally with, or coupled to a particular plate 24 while gusset 64 can be coupled to, or formed integrally with, another particular plate 24. Hinge 61 can pivotably couple plates 24 together while allowing relative rotation of plates 24 about a superior-inferior axis, a medial-lateral axis, and an anterior-posterior axis or combinations thereof. Hinge 61 can therefore allow plates 24 to follow anatomical movements of spine 10 as spine 10 flexes, extends, twists, rotates, etc.

FIG. 8 also illustrates hinge 68 of one embodiment which can include spring 68 coupled to, or formed integrally with, plates 24. Spring 70 can be shaped, dimensioned, etc. to provide a spring constant selected by surgical personnel to provide the patient desired amounts of restraint against movement (or desired freedom of movement). Thus, spring 70 can pivotably couple plates 24 and can allow plates 24 to follow anatomical movements of spine 10 as spine 10 extends and flexes. Spring 70 can be a helical spring, a conical spring, etc. coupled to, or formed integrally with plates 24, without departing from the scope of the disclosure. Hinges 26 can be sealed with a jacket if desired. Types of hinges 26 other than pin type hinges 49, pin and socket type hinges 55, ball and socket type hinges 61, and spring hinges 68 (see FIG. 9) can be used without departing from the scope of the disclosure.

FIG. 9A illustrates one embodiment of a piston 71 and cylinder 73 coupling plates 24 to each other. Piston 71 can translate relative to cylinder 73 within cylinder 73 thereby allowing spine 10 to extend and flex. Piston 71 and cylinder 73 can be straight in which case spine 10 can be constrained to only extend and flex. In some embodiments, piston 71 and cylinder 73 can be curved, with corresponding radii of curvature so that piston 71 can translate along a curved path to allow spine 10 to flex and extend and to allow vertebrae (to which plates 24 can be attached) of spine 10 to rotate relative to each other. Cylinder 73 can be filled with a viscous fluid to damp movements of piston 71 and spine 10. In some embodiments, cylinder 73 can be filled with a fluid with a selected compressibility such that as piston 71 translates toward one end or another of cylinder 73, the force required to translate piston 71 increases by a selected amount, at a selected rate, etc. Thus, cylinder 73 can be filled with air, saline solution, etc. In some embodiments, a spring can be included within cylinder 73 to provide a selected amount of restraint against translation of piston 71. Piston 71 can therefore be biased to return to a user selection position relative to spine 10 in the absence of outside forces (such as those exerted on plates 24 by the patient). Piston 71 and cylinder 73 can be configured (with springs, fluid fillings, etc.) to limit translation of piston 71 relative to cylinder 73 within a selected range. In some embodiments, piston 71 and cylinder 73 (whether straight or curved) can be combined with other types of hinges such as those illustrated in FIG. 8.

For instance, with reference now to FIG. 9B, FIG. 9B illustrates ball and socket hinge 61, plates 24, cylinder 72, and piston 74 of one embodiment. Cylinder 72 can be coupled to, or formed integrally with, a particular plate 24. Piston 74 can be coupled with the other plate 24 via ball 62 and gusset 64. Cylinder 72 can contain a fluid and appropriate bleed orifices to allow piston 74 to translate along cylinder 72 while damping relative motions of plates 224 and affected vertebra 18. Cylinder 72 can include travel stops 76 to prevent piston 74 from traveling beyond selected points relative to cylinder 72. Thus, cylinder 72 and piston 74 can allow plates 24 to translate along a superior-inferior axis relative to vertebrae 12. Piston 74 can include travel stops 78 positioned to contact gusset 64 should ball 62 and gusset 64 allow relative rotation between plates 24 beyond a user selected amount. Together, cylinder 72, piston 74, ball 62, and gusset 64, can allow plates 24 to follow anatomical movements of spine 10 as spine 10 flexes, extends, twists, rotates, stretches, and compresses. Travel stops 76 and 78 can limit relative motion between plates 24 as may be desired during such movements of spine 10.

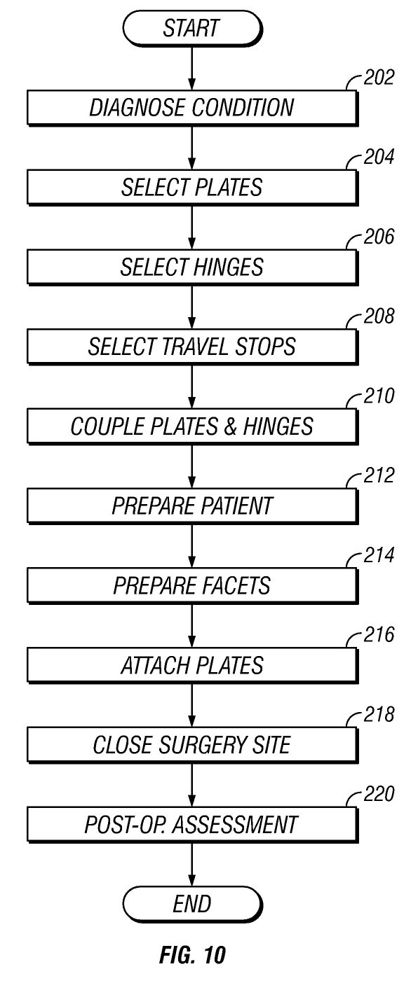

With reference now to FIG. 10, FIG. 10 illustrates one embodiment of a method for stabilizing spine 10. At step 202, method 200 can include diagnosing patient symptoms including conducting interviews of the patient, palpating affected regions of spine 10, analyzing certain ranges of motion of the patient, and imaging affected areas of spine 10 with X-ray, MRI, CT, CAT, etc. imaging techniques. More specifically, the particular location along spine 10 at which the injury or degradation may have occurred can be determined. Relevant anatomical features including angles a1 and a2 of transverse processes 17 (or vertebral facets 18) of affected vertebrae 12 can be determined from various images gathered during diagnosis of patient symptoms. Plates 24 can be selected from a variety of plates 24 having differing heights, widths, thicknesses, etc. Plates 24 can include plates 24 with, and without, adapters 228 and 230 of varied angles a1 and a2. In selecting plates 24, consideration can be given to whether to attach plates 24 to transverse processes 17 or vertebral facets 18. Consideration can be given to whether vertebral facets 18 should be totally or partially removed to create posterior attachment surfaces 23 for plates 24. Thus, plates 24 can be selected at step 204 as desired by surgical personnel.

Hinge 26 may be selected at step 206 from pin type hinges 49, pin and socket type hinges 55, ball and socket type hinges 61, and spring hinges 68 (see FIG. 9), etc. as desired by medical personnel. Hinges 49, 55, 61, and 68 from which the selection can be made can include hinges of varying geometries, mechanical properties, etc. At step 208, travel stops can be selected for use with selected hinge 26. Spinal stabilization system 22 can be assembled by appropriate personnel at step 210. More particularly, hinge 26 can be used to couple plates 24 together.

At a selected time, surgical personnel can prepare the patient for surgery at step 212. The patient can be placed on an operating table or surface in a face down position when it is desired to attach spinal stabilization system 22 to spine 10 using a posterior approach. The patient can be anesthetized as desired by surgical personnel and an incision can be made in the proximity of affected vertebrae 12 of spine 10. Soft tissue can be distracted from vertebrae 18. Surgical personnel can evaluate vertebrae 12, intervertebral discs 14, transverse processes 17, vertebral facets 18, and spinal stabilization system 22 to confirm selection of appropriate plates 24 and hinges 26. Surgical personnel can evaluate vertebrae 12, intervertebral discs 14, transverse processes 17, vertebral facets 18, and spinal stabilization system 22 to confirm decisions relating to removing (or not removing) vertebral facets 18.

When desired, vertebral facets 18 can be removed totally, or partially, as desired by surgical personnel at step 214. In some embodiments, vertebral facets can be partially removed leaving the exposed surfaces reflecting angles a1 and a2 of plates 24 selected by at step 204. When only one level of spine 10 is to be stabilized, a particular plate 24 can be attached to vertebral facet 18, exposed surfaces 23, or transverse processes 17 at step 216. The other plate 24 can be attached to its corresponding vertebral facet 18 (or transverse processes 17). Surgical personnel can evaluate attached spinal stabilization system 22 to determine its mechanical integrity and functioning and make adjustments accordingly.

At step 216, when more than one level of spine 10 is to be stabilized, plates 134 and 136 of multiple level spinal stabilization system 122 (see FIGS. 4 and 5) can be aligned using mating keys 140. Surgical personnel can attach plates 124 to vertebrae 12 using suitable attachment device(s) such as a bone screw at step 216. Additional plates 124 can be attached to appropriate transverse processes 17 or vertebral facets 18 at step 216 until spinal stabilization system 22 is attached to spine 10. Surgical personnel can evaluate attached spinal stabilization system 22 to determine its mechanical integrity and functioning and make adjustments accordingly.

When desired, at step 218, surgical personnel can close the surgical site including returning distracted soft tissues to their original location, closing the incision made in the proximity of spine 10, etc. Medical personnel can conduct post-operative evaluations of spinal stabilization system 22 including conducting interviews of the patient, palpating affected regions of spine 10, analyzing certain ranges of motion of the patient associated with spine 10, and imaging affected areas of spine 10 with X-ray, MRI, CT, CAT, etc. imaging techniques at step 220.

Embodiments provide spinal stabilization systems in which hinge mechanisms are located more proximal to the vertebral body than the attachment points. Various embodiments locate hinge mechanisms closer to the centers of rotation of adjacent vertebrae than heretofore possible. Embodiments attach directly to the vertebral body. Improved range of motion for patients treated with spinal stabilization systems can be provided by embodiments. Various embodiments reduce the force patients exert to move in manners which cause their spines to flex, extend, rotate, twist, etc. while reducing forces exerted on their spines by the spinal stabilization systems.

In the foregoing specification, specific embodiments have been described with reference to the accompanying drawings. However, as one skilled in the art can appreciate, embodiments of the anisotropic spinal stabilization rod disclosed herein can be modified or otherwise implemented in many ways Without departing from the spirit and scope of the disclosure. Accordingly, this description is to be construed as illustrative only and is for the purpose of teaching those skilled in the art the manner of making and using embodiments of an anisotropic spinal stabilization rod. It is to be understood that the embodiments shown arid described herein are to be taken as exemplary. Equivalent elements or materials may be substituted for those illustrated and described herein. Moreover, certain features of the disclosure may be utilized independently of the use of other features, all as would be apparent to one skilled in the art after having the benefit of this description of the disclosure.

1. A posterior dynamic spinal stabilization system comprising:
a first plate being shaped to conform to a posterior surface of a first vertebra of a spine;
a second plate being shaped to conform to a posterior surface of a second vertebra of the spine, the plates to be attached to the respective vertebral surfaces; and
a hinge pivotably coupling the first plate to the second plate, the vertebrae having a center of rotation about which the vertebrae rotate relative to each other when the spine flexes or extends, the hinge being positioned relative to at least one of the plates to be generally adjacent the center of rotation when the plates are attached to the respective vertebral surfaces.

2. The system of claim 1 wherein the hinge includes a ball and socket.

3. The system of claim 1 wherein the hinge includes a pin and a pin hole.

4. The system of claim 1 wherein the surfaces are on facets of the vertebra.

5. The system of claim 1 wherein the surfaces are surfaces of the vertebral bodies which are exposed when the facets were removed.

6. The system of claim 1 further comprising a first key on the first plate adapted to mate with a second key on a plate of another system wherein multiple systems may be used to stabilize multiple levels of the spine.

7. The system of claim 6 wherein, when the keys are mated, the keys overlap each other and define an aperture for an attachment device to attach the plates to one of the surfaces.

8. The system of claim 1 further comprising a piston coupled to the hinge and one of the plates.

9. The system of claim 1 further Comprising a travel limit of the piston.

10. The system of claim 1 wherein the hinge is a spring.

11. The system of claim 1 further comprising a jacket sealing the hinge.

12. A method of dynamically stabilizing a spine comprising:
selecting a first plate of a posterior spinal stabilization system from a plurality of plates, each plate being shaped to conform to a posterior surface of a vertebra of the spine;
selecting a second plate from the plurality of plates;
causing the first plate to be pivotably coupled to the second plate by a hinge;
selecting a position on a first posterior surface of a first vertebra to attach the first plate such that, when the first plate is attached to the first surface, the hinge will be positioned generally adjacent to a center of rotation of the vertebrae about which the vertebrae rotate when the spine flexes or extends;
attaching the first plate to the first surface at the selected position; and
attaching the second plate to a second posterior surface of the second vertebra.

13. The method of claim 12 further comprising removing a facet from the first and second vertebrae to expose the first and second surfaces.

14. The method of claim 12 wherein the first and second surfaces are on facets of the first and second vertebrae respectively.

15. The method of claim 12 further comprising aligning a first key on the first plate with a second key on a third plate and attaching the third plate to the first vertebra.

16. The method of claim 15 further comprising attaching the first and third plates to the first vertebra using a pair of apertures defined by the first and second keys.

17. The method of claim 12 further comprising selecting the hinge from the group consisting of a hinge including a ball and socket, a hinge including a pin and pin hole, and a spring.

18. The method of claim 12 further comprising selecting a piston with which to couple one of the plates to the hinge.

19. The method of claim 18 further comprising selecting a travel limit for the piston.

20. A posterior dynamic spinal stabilization system comprising:
a first plate being shaped to conform to a facet a first vertebra of a spine;
a second plate being shaped to conform to a facet of a second vertebra of the spine, the plates to be attached to the respective facets;
a hinge including a pin and a pin hole and pivotably coupling the first plate to the second plate, the vertebrae having a center of rotation about which the vertebrae rotate relative to each other when the spine flexes or extends, the hinge being positioned relative to at least one of the plates to be generally adjacent the center of rotation when the plates are attached to the respective vertebral surfaces; and
a travel limit positioned to limit the travel of the plates relative to each other about the hinge.
